(19) United States
(12) Patent Application Publication
(10) Pub. No.: US 2022/0049283 A1
(43) Pub. Date: Feb. 17, 2022

(54) Method of detecting SARS-CoV-2 cleavage targeted proprotein convertase and facilitated protease activity

(71) Applicants: Weiwei Li Li, Woodbury, NY (US); Jessica Mliu Li, Woodbury, NY (US); William Lei Lee, Mineola, NY (US)

(72) Inventors: Weiwei Li Li, Woodbury, NY (US); Jessica Mliu Li, Woodbury, NY (US); William Lei Lee, Mineola, NY (US)

(21) Appl. No.: 16/947,760

(22) Filed: Aug. 14, 2020

(51) Int. Cl. C12Q 1/37; G01N 33/543; G01N 21/78

(52) U.S. Cl. CPC C12Q 1/37; G01N 33/54306; G01N 2800/26; G01N 2800/50; G01N 21/78

(57) ABSTRACT

The present invention provides the methods of detecting the cleavage activity of PCs and proteases that are in purified form or from a biological sample and target to SARS-CoV-2 spike protein cleavage.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

### Field of the Invention

This invention is related to methods useful for detecting activity of proprotein convertases (PC) and proteases that are in purified form or from a biological sample, and target to SARS-CoV-2 spike protein cleavage.

### Description of the Related Art

The recent COVID-19 pandemic is caused by SARS-CoV-2, a new member of the same coronavirus family that caused SARS and MERS. So far, there are no vaccines or effective drugs that can be used for prevention and treatment of COVID-19. It has been observed that in the same exposure environment to SARS-CoV-2, some people become infected while others do not. Those infected with SARS-CoV-2 can either display severe illness or be asymptomatic/mildly symptomatic.

It was found that the SARS-CoV-2 spike (S) glycoprotein harbors a furin cleavage site at the boundary between the S i/S₂ subunits, which could be cleaved by furin and/or furin-like PCs secreted from host cells and bacteria in the airway epithelium. Unlike SARS-CoV, cell entry of SARS-CoV-2 needs to be pre-activated by furin and/or furin-like PCs, reducing its dependence on target cell proteases for entry. The cleavage activation of S-protein is well demonstrated to be essential for SARS-CoV-2 spike-mediated viral binding to the host ACE2 receptor, cell-cell fusion, and viral entry into human lung cells. It was also observed that other viruses containing a furin cleavage site, such as H5N1, displayed increased replicates and developed higher pathogenicity.

As shown in FIG. 1, the complete SARS-CoV-2 furin cleavage site has been characterized as a 20 amino acid motif running from the P14-P6′ region, with one core region SPRRAR|SV (eight amino acids, P6-P2′) and two flanking solvent accessible regions (eight amino acids, P7-P14, and four amino acids, P3′-P6′). The core region is very unique as its P2 and P3 positions are positively charged (Arg) and hydrophobic (Ala) residues, respectively, which allow this site to not only be cleaved by serine protease furin or furin-like PCs, but also permit cleavage efficiency to be facilitated by other serine proteases targeting mono- and dibasic amino acid sites such as matriptase, kallikrein 1 (KLK1), human airway trypsin (HAT), and TMPRSS2. Furthermore, a serine at P6 could also highly increase the cleavage efficiency, causing increased viral replication, unrestricted organ tropism, and virulence and mortality rate as proven in H5N1 infection studies in mice.

Furin and furin-like PCs, such as PC5/6A and PACE4, are proven to be cleavage region sequence-specific and these PCs exhibit widespread tissue distribution. With the unique furin cleavage site in SARS-CoV-2, such distribution may explain why COVID-19 causes damage in multiple organs. Thus, the importance of measuring SARS-CoV-2 S1/S2 site cleavage targeted PC or facilitating protease activity is emphasized by the fact that PC-based SARS-CoV-2 S1/S2 cleavage increases SARS-CoV-2 entry into cells and its replication, and eventually develops higher pathogenicity of COVID-19. Such SARS-CoV-2 cleavage targeted PC and facilitated protease activity detection would b e great; u beneficial in predicting differential COVID-19 susceptibility and pathogenicity as well as developing SARS-CoV-2 cleavage inhibitors.

The activity detection of PCs and facilitated proteases is generally based on hydrolysis of a peptide substrate by the enzymes through specific interactions between catalytic sites of the enzymes and complementary amino acid sequences through covalent bond formation, electrostatic attraction, hydrogen bonding, or van der Waal's forces. The activity that is proportional signal intensity generated in hydrolysis can then be measured through various detectors such as optical density reading, fluorescence electrochemical detection, spectrometry, fluorescence resonance energy transfer (FRET) assays, surface-enhanced Raman scattering (SERS) assays, and surface plasmon resonance (SPR) assays. Currently there are a few of methods available for measuring activity of proteases including furin and furin-like PCs. However, there are no methods available for measuring SARS-CoV-2 cleavage targeted PC or related protease activity and inhibition, and there is a need to develop such detection methods.

### BRIEF SUMMARY OF THE INVENTION

The present invention provides the methods of detecting the activity of PCs and proteases that are in purified form or from a biological sample, and target to SARS-CoV-2 spike protein cleavage, comprising the steps of:

1) Generate an amino acid substrate containing SARS-CoV-2 S1/S2 sequences that can be cleaved by SARS-CoV-2 cleavage targeted PCs and facilitated proteases;
2) Label the substrate at N-terminal with an affinity moiety and/or at C-terminal with another different affinity moiety;
3) Binding of the substrate to a solid-phase carrier through the interaction of labeled affinity moiety to a binding partner coated on the solid-phase carrier, or if the substrate is dual labeled at both terminal, binding of the substrate to the solid-phase carrier through one of the affinity moiety at N-terminal or C-terminal, allowing another one of the affinity moiety at N-terminal or C-terminal to be unbound;
4) If the substrate is dual-labeled, detection of the unbound affinity moiety in the presence or absence of SARS-CoV-2 cleavage targeted PC or related proteases with indicator molecules that are able to interact with unbound affinity moiety; or if the substrate is single-labeled, detection of the sequence of unlabeled N-terminal or C-terminal with the sequence-specific recognized molecules.
5) Fluorescent or color development of the indicator molecules or the sequence-specific recognized substances and quantification of fluorescent or color intensity.

Thus the invention allows for a rapid detection of SARS-CoV-2 cleavage targeted PC or related protease activity to be achieved. The invention is based on the finding that the SARS-CoV-2 specific substrate containing a certain length of SARS-CoV-2 S1/S2 boundary sequence including furin site allows the substrate to be cleaved not only by PCs but also by certain proteases. The invention is also based on the finding that affinity moiety labeled substrate can more rapidly and conveniently bind to and have retention on the solid-phase carrier at appropriate temperature. The invention is further based on the finding that the detection of SARS-CoV-2 cleavage targeted PC or related protease activity can be quantitatively achieved through recognition of unbounded affinity moiety by indicator molecules or the sequence of N-terminal or C-terminal by sequence-specific recognized molecules followed by color or fluorescence development and measurement. Therefore the method presented in this invention significantly overcomes the weaknesses existing in the prior technologies and enables SARS-CoV-2 cleavage targeted PC or facilitated protease activity to be detected rapidly and efficiently.

### BRIEF DESCRIPTION OF THE DRAWINGS

FIG. 1 Shows structure of SARS-CoV-2 spike protein and the SARS-CoV-2 S1/S2 boundary sequence contained in the substrate.

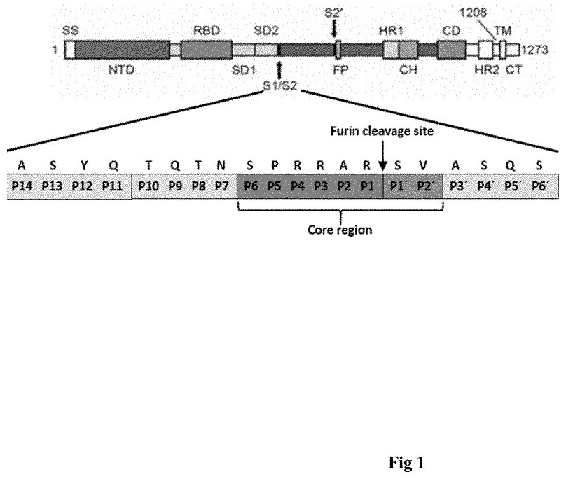

FIG. 2 shows a diagram of the rapid process for detecting SARS-CoV-2 cleavage targeted PC or facilitated protease activity. The process involves: (1) binding of the substrate on the plastic well through the labeled affinity moiety-binding partner coated on the plastic well; (2) cleavage of the substrate and removal of cleaved substrate part by PCs or facilitated proteases; (3) the detection of the unbound affinity moiety with indicator molecules or the detection of the sequence of unlabeled N-terminal or C-terminal with the sequence-specific recognized molecules; and (4) fluorescent or color development of the indicator molecules or the sequence-specific recognized substances and quantification of fluorescent or color intensity. The more the substrate is cleaved, the lower the signal that will be generated. Thus, the cleavage activity is inversely proportional to the signal intensity.

FIG. 3 shows the cleavage activity and inhibition of purified PC or facilitated proteases with a synthesized SARS-CoV-2 specific peptide as the substrate using the method of this invention. The experiment was carried out as described in Example 1. Enzyme Concentration (ng/well): Furin: 10 ng; Plasmin: 50 ng; Trypsin: 50 ng; HAT: 200 ng; Protease inhibitor (aprotinin): 500 ng.

FIG. 4 shows the sensitivity of the method of this invention in detection of SARS-CoV-2 cleavage targeted PC or facilitated protease activity with use of the synthesized SARS-CoV-2 specific peptide as the substrate. The experiment was carried out as described in Example 2.

FIG. 5 shows the cleavage activity of the biological samples with synthesized SARS-CoV-2 specific peptide as the substrate using the method of this invention. The experiment was carried out as described in Example 3. Human nasal and oral swab sample: released into 300 μl of PC assay buffer and 30 μl of sample solution was used for assay. Human serum sample: 5 μl.

FIG. 6 shows the cleavage activity of purified PC or facilitated proteases with use of complete trimeric form of full-length SARS-CoV-2 spike protein as the substrate using the method of this invention. The experiment was carried out as described in Example 4. Enzyme concentration (ng/well): Furin: 10 ng; Trypsin: 50 ng; Protease inhibitor (aprotinin): 500 ng.

### DETAILED DESCRIPTION OF THE INVENTION

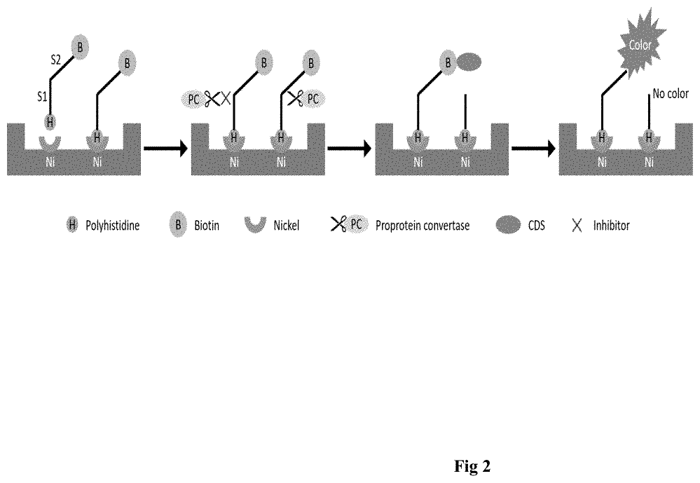

The present invention provides a method for rapidly detecting SARS-CoV-2 cleavage targeted PC or facilitated protease activity through the binding of the substrate on the solid-phase carrier followed by detection of (a) the unbound affinity moiety labeled on the substrate with indicator molecules; or (b) the detection of the sequence of unlabeled N-terminal or C-terminal with the sequence-specific recognized molecules. A basic outline of the method presented in this invention is described in FIG. 2. This method is particularly useful for rapidly completing SARS-CoV-2 cleavage targeted PC or facilitated protease activity assay in a short time. This method is also particularly useful for detecting SARS-CoV-2 cleavage targeted PC or facilitated protease activity in a high throughput format.

According to the method of this invention, the substrate could be a synthesized peptide containing a certain length of SARS-CoV-2 S1/S2 boundary sequence including furin cleavage motif. The peptide can be consisted of minimal 8 amino acids and maximal 500 amino acids in the length, which contains at least a SARS-CoV-2 specific core region (P4-P2′) of furin cleavage motif, or a full SARS-CoV-2 specific furin cleavage motif. This full SARS-CoV-2 specific furin cleavage motif has 20 amino acid motif running from the P14-P6′: Ala-Ser-Tyr-Gln-Thr-Gln-Thr-Asn-Ser-Pro-Arg-Arg-Ala-Arg-Ser-Val-Ala-Se-Gln-Ser (ASYQTQTNSPRRARSVAS QS) (SEQ ID 1). The peptide may contain multiple SARS-CoV-2 specific core regions of furin cleavage motif, or multiple full SARS-CoV-2 specific furin cleavage regions in certain embodiments. The substrate could be also a recombinant full length SARS-CoV-2 fusion protein (receptor-binding subunit 51 and membrane fusion subunit S2) that may reflect a complete tertiary structure of the protein they are usually embedded in as the interaction between the SARS-CoV-2 targeted PCs/facilitated proteases and fusion protein might be impacted by the tertiary structure of the fusion protein.

According to the method of this invention, the affinity moiety of the substrate and affinity partner coated on the solid-phase carrier are two halves of a binding pair. This binding pair could be selected from biotin and streptavidin, neutravidin, or captavdin; a nickel and polyhistidine; an antigen and antibody or antigen binding fragment; an IgG immunoglobulin domain and protein A or protein G; a receptor molecule and a ligand, a GST and GSH; and complementary nucleotide sequences. In particular embodiments of the invention, the binding pair consists of biotin and streptavidin, nickel and polyhistidine and/or antigen and antibody. In a further embodiment of the invention, the antigen can be a specific amino acid sequence and Fc fragment of IgG. The antibody can be monoclonal or polyclonal and refers to different types such as chimeric antibodies humanized antibodies, single domain nanobodies, F(ab′)₂ fragments, Fab fragments, Fv fragments, and sFv fragments.

The substrate can then be bound to the affinity partner-coated solid-phase carrier by using appropriate buffers. The solid-phase carrier includes but is not limited to polystyrene plastic, glass, silica and metal. The carrier could be in various sizes and forms including, but not limited to beads in size of 10 nm-100 um, strip with 8-wells-12-wells, microplate with 6 wells-1516 wells, microscopic slide with or without wells, and microarray slide with or without wells. Preferably, the plastic microplate or strips are more suitable to be used in the method of this invention, as these carriers are easily handled in a rapid and high throughput format. The appropriate binding buffers include but are not limited to bicarbonate buffer, phosphate buffer, glycine/NaOH buffer, Tris-sodium buffer and HEPES-sodium buffer. The microplate or strip is incubated at 37° C. with or without humidity for 90 min. The beads are incubated at 37° C. with or without humidity for 90 min. For microscopic or microarray slides, the substrate is first mixed with binding buffer, and 0.2-4 ul of the mixed solution, depending on the required number of spots, is added to each spot area. The slide can be incubated at 37° C. without humidity for 45 min to dry the spotted area. The substrate amount to be bound can be from 1 to 200 ng, preferably, from 5 to 50 ng, and more preferably, 20 ng. A 10 ng of substrate amount would ensure the sufficient uncleaved substrate amount to be detectable while still allowing high specificity to be achieved.

According to the method of the invention, the SARS-CoV-2 targeted PCs and facilitated proteases can be recombinant proteins with enzyme activity or a biological sample containing such PCs and facilitated proteases. These PCs or facilitated proteases must be capable of cleaving the SARS-Cov-2 specific substrate and therefore producing at least two parts of the cleavage region of the substrate, at least one part of which remains bound to the binding partner on the solid-phase carrier. These PCs and facilitated proteases may include but are not limited to proprotein convertases such as Turin, PC1/3, PC2, PACE4, PC5/6A, PC7, SKI-1/S1P and PC9; type II transmembrane serine proteases such as human air trypsin (HAT), TMPRSS2, TMPRSS4, TMPRSS11A, matriptase; cysteine proteases such as cathepsin A, cathepsin B and cathepsin L; thrombin-like protease such as plasmin, thrombin, and tissue activating plasminogen.

According to the method of the invention, the biological sample can be from various sources including fluids such as serum, plasma, saliva, urine, milk, tears from the eye, ascites fluid, peritoneal fluid and, amniotic fluid. In some embodiments, the sample could be a swab sample from the nasal cavity, oral cavity, nasopharyngeal, vagina, and endocervix. In further embodiments, the sample can be solid tissues collected from various organs and can be cultured cells from laboratories. The liquid and solid samples can be collected by an appropriate methods or standard procedure, pre-treated to allow for immediate test with the method of this invention. The sample may also be stored at an appropriate temperature for preserving the sample, for example stored at 4° C., −20° C. or −80° C. based on the sample types for a given length of time before test with the method of this invention.

After the substrate is bound to a solid-phase carrier, the solid-phase carrier is washed with the washing buffer comprising tris-saline and 0.05% tween-20, preferably comprising phosphate saline and 0.1% tween-20. Once the washing is completed, the purified enzymes or biological samples can be added and incubate with substrate for a given length of time in the presence of assay buffer. The assay buffer may include bicarbonate buffer, phosphate buffer, glycine/NaOH buffer, Tris-HCl buffer and HEPES buffer. In some embodiments of this invention, the assay buffers may contain sodium salt, magnesium salt and calcium salt. In further embodiments, the assay buffers may contain detergents such as NP-40, triton-X 100, tween-20 and tween-80.

According to the method of this invention, after incubation of the substrate with the purified enzymes or biological samples, the solid-phase carrier is washed with the washing buffer again. If the substrate is a synthesized peptide, the binding partner-specific to unbound affinity moiety of the substrate is added to the solid-phase carrier. The binding partner may include biotin-bound streptavidin, neutravidin or captavdin; nickel-bound histidine and GST-bound GSH. The binding partner should be conjugated with the reporter molecules to form indicator molecules that are typically capable of signal generation or production. These reporter molecules may include enzymatic label molecules such as HRP and AP, or fluorescent label molecules such as cy3, cy5, FITC, or gold label molecules, or quantum dot label molecules. The final concentration of the indicator molecules added to the solid-phase carrier should be 0.1-1 μg/ml. If the substrate is a recombinant SARS-CoV-2 spike protein, an antibody specific against N-terminal sequence or C-terminal sequence of the protein as the binding partner can be added to the solid-phase carrier. The antibody specific to the said sequences may include mouse monoclonal IgG, rat monoclonal IgG, rabbit polyclonal IgG, goat polyclonal IgG and sheep polyclonal IgG. The antibody can be unconjugated or conjugated with enzymatic label molecules such as HRP and AP, or fluorescent label molecules such as cy3, cy5, FITC, gold label molecules, or quantum dot label molecules to form an indicator molecule. The final concentration of the antibody added to the plastic carrier should be 0.5-1 μg/ml.

The solid-phase carrier is incubated at room temperature or 37° C. for a given length of time after adding the indicator molecules. After incubation, the solid-phase carrier is washed with the wash buffer for several times. If the indicator molecules or conjugated antibody is used, a colorimetric or fluorescent development can be directly carried out followed by signal measurement. If the unconjugated antibody is used, a secondary anti-mouse, anti-rabbit, anti-goat, or anti-sheep antibody conjugated with reporter molecules is added to the solid-phase carrier. The final concentration of the secondary antibody can be from 0.01 μg/ml to 0.5 ug/ml. The label molecules, depending on the requirement of assay, include but are not limited to horse radish peroxidase (HRP), alkaline phosphotase (AP), biotin, fluorescein (FITC), Cy3, Cy5, rhodamine, dynabeads, texas red, Alexa fluor, BODIPY, captivate ferrofluid, cascade blue, beta-lactamase, marine blue, nanogold, Oregon green, pacific blue, and quantum dot. After washing with wash buffer for several times again, the uncleaved substrate can be quantitatively detected through the colorimetric or fluorescent development. For colorimetric development, the solution containing color-forming substance is added to react with enzymatic label molecules such as HRP or AP to yield blue solution or deposit. Other suitable color-forming substrates will be apparent to persons skilled in the art. For fluorescent measurement, fluorescent intensity is directly directed with fluorescent spectrophotometer, fluorescent scanner, or fluorescent microscope.

According to the invention, the peptide as SARS-CoV-2 specific substrate can be synthesized through a commercially available service or generated by a solid phase peptide synthesis method using Fmoc-chemistry, which allows for synthesizing of peptides with a length up to 50 amino-acids. In specific embodiments, a longer peptide can be synthesized by fragment synthesis and chemical ligation technologies, which allows for synthesizing peptides with a length up to 150 amino-acids. A full length of recombinant SARS-CoV-2 spike protein as substrate can be generated through a commercially available service or generated by a mature recombinant protein production method with the following major steps: (1) generating the cDNA and creating the expression clone; (2) cloning; (3) expressing the protein in a bacterial or mammalian system; (4) small-scale test expression; and (5) purifying recombinant protein. For attachment of the affinity moiety to the substrate, the services for the peptide and protein labeling with the affinity moieties are commercially available. The substrate labeling with biotin or polyhistidine may also be generated with several methods. These methods for biotin labeling include but not are limited to enzymatic conjugation by AviTag or Acceptor Peptide using biotin ligase; primary amine conjugation by amine-reactive biotinylation agents N-hydroxysuccinimide (NHS) or by carbodiimide crosslinker; glycoprotein conjugation by modifying the carbohydrate residues to aldehydes, and then reacting with hydrazine- or alkoxyamine-based biotinylation reagents. For attachment of the polyhistidine to the substrate, a polyhistidine coupling method can be used for the poly-his tagged peptide with DIC/HOBt as reagent for activating the —COOH group of histidine. A his-tag fusion vector method can be used for the recombinant protein by adding his-tag via insertion of the genomic RNA encoding the SARS-CoV-2 spike protein in a vector that has the tag ready to fuse at the C-terminus, or adding his-tag using primers containing the tag, after a PCR reaction the tag gets fused to the N-terminus of the gene.

In certain embodiments of the invention, a polyclonal or monoclonal antibody specific against the N-terminal sequence or C-terminal sequence of the substrate, respectively, can also be commercially available or generated by using mature antibody production methods: (1) Preparation of the antigen-KLH conjugates. KLH may be modified with 3-sulfo-N-hydroxysuccinimide ester sodium salt before conjugation. The conjugates of KLH-antigen can be identified by ultraviolet spectrophotometry; (2) Injection of KLH-antigen into rabbits or mouse. Injections of the antigen are given in multiple sites to stimulate the best immune. For the polyclonal antibody, the rabbits are boosted at 3 to 4 week intervals until peak antibody titers are reached (6-8 re-immunizations). And blood is collected and clotted. The clotted blood is then refrigerated for 24 hours before the serum is decanted and clarified by centrifugation. ELISA test of antibody titers and affinity purification are then performed. For the monoclonal antibody, cells from the spleen of the immunized rabbit or mouse are collected and are fused with cultured myeloma cells to yield hybridoma cells. Then hybridoma cells that produce antibodies in culture are grown and the desired monoclonal antibody is screened and purified.

According to the invention, all of the components for sample collection, solid-phase carrier, substances for substrate binding, and signal detection are commercially available. This invention also provides a kit containing all components required for rapid detection of SARS-CoV-2 targeted PC and facilitated protease cleavage activity in a multi-well microplate/strip format. The kit includes: (a) a cleavage activity indicator substance conjugated with reporter molecules for signal production; (b) a microwell strip or microwell plate coated with substrate; (c) the PC and facilitated protease assay buffer; (d) the concentrated washing buffer comprised of phosphate-saline and surfactants; (e) a colorimetric development solution containing color-forming substrate specific for HRP or a fluorometric development solution containing fluorescent-forming substance; (f) a positive control of protease cleavage; and (g) an instruction sheet for conducting an assay according to the method of this invention. In one embodiment, the kit further comprises of selected components to meet the requirements for using different measurement equipments.

It is unexpected that the SARS-CoV-2 spike protein containing a specific furin region can be cleaved not only by furin or furin-like proprotein convertases but also by other serine proteases targeting to mono- and/or dibasic cleavage site in this region. It is also unexpected that the dual-labeled SARS-CoV-2 specific substrate enables the detection to be highly sensitive so that PC cleavage activity can be detected in a tiny amount of samples such as nasal swab samples. Further, it has been discovered that the use of the method of this invention is able to drastically reduce the cost and time required for detecting SARS-CoV-2 targeted PCs and facilitated proteases. It has been also discovered that the use of the method of this invention is able to allow SARS-CoV-2 targeted PC and facilitated protease cleavage activity assay to be much easer and more convenient than currently used methods, as the method based on this invention can be carried out with regular equipment such as a microplate reader or even only with a smartphone. It has been further discovered that the use of the method of this invention enables the SARS-CoV-2 targeted PC and facilitated protease cleavage activity assay to be performed in a high throughput format with high specificity, and to be completed with excellent reproducibility.

The method of this invention for SARS-CoV-2 targeted PC and facilitated protease cleavage activity assay is further illustrated in the following examples:

### Example 1

The experiment was carried out to detect the cleavage activity and inhibition of purified PCs or facilitated proteases with synthesized SARS-CoV-2 specific peptide as the substrate using the method of this invention.

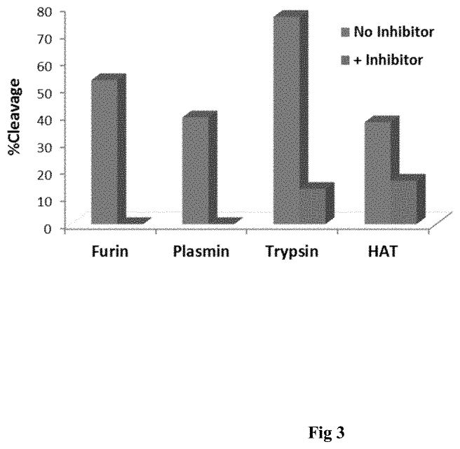

In this experiment, polystyrene 8-well strip microplate was coated with nickel (Ni-NTA). A SARS-CoV-2 specific substrate is tagged with polyhistidine at N-terminal and biotin at C-terminal and bound onto microplate wells through histidine/Ni-NTA at its N-terminal at the concentration of 10 ng/well. The strips were then incubated at 37° C. for 25 min. After wash with PBS-T for 3 times, the purified proprotein convertase furin (New England Biolabs), serine protease trypsin (Sigma), plasmin (Sigma), and HAT (Sino Biological) were diluted to an indicated concentration with PC assay buffer consisted of sodium phosphate, sodium chloride, and cesium chloride and were added into the wells for incubation for 25 min. The wells were washed with PBS-T for 4 times. The cleavage of the substrate at S1/S2 site will remove the S2 part (C-terminal) of the substrate after washing, 100 ul of streptavidin-HRP at 1:5000 dilution was added into the wells for 15 min incubation. After washing 4 times, 100 μl of the color development solution containing TMB was added into the wells and wells were observed for 2-10 min for blue color appearance. The 1 M HCl or H₂504 solution was added to stop the color development and the optical density was measured with a microplate reader at a wavelength of 450 nm or measured with Spotxel® Reader installed on a smartphone. The results are shown in the FIG. 3.

### Example 2

The experiment was carried out to determine the sensitivity of the method of this invention in detection of SARS-CoV-2 cleavage targeted PC or facilitated protease activity with use of the synthesized SARS-CoV-2 specific peptide as the substrate.

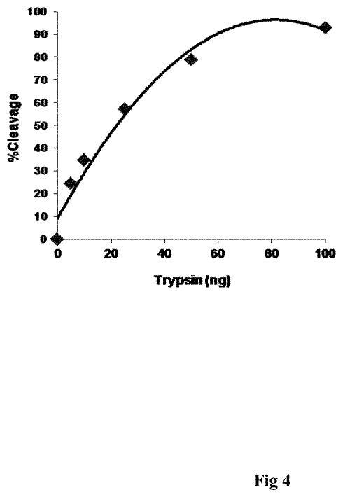

In this experiment, polystyrene 8-well strip microplate was coated with nickel (Ni-NTA). A SARS-CoV-2 specific substrate is tagged with polyhistidine at N-terminal and biotin at C-terminal and bound onto microplate wells through histidine/Ni-NTA at its N-terminal at the concentration of 10 ng/well. The strips were then incubated at 37° C. for 25 min. After wash with PBS-T for 3 times, the purified serine protease trypsin was diluted to different concentrations with PC assay buffer consisted of sodium phosphate, sodium chloride and cesium chloride and added into the wells for incubation for 25 min. The wells were washed with PBS-T for 4 times. The cleavage of the substrate at S1/S2 site will remove the S2 part (C-terminal) of the substrate after washing, 100 ul of streptavidin-HRP at 1:5000 dilution was added into the wells for 15 min incubation. After washing 4 times, 100 μl of the color development solution containing TMB was added into the wells and wells were observed for 2-10 min for blue color appearance. The 1 M HCl or H₂SO4 solution was added to stop the color development and the optical density was measured with a microplate reader at a wavelength of 450 nm or measured with Spotxel® Reader installed on a smartphone. As shown in FIG. 4, the cleavage activity generated from as low as 10 ng of trypsin can be detected.

### Example 3

The experiment was carried out to examine the cleavage activity of the biological samples with synthesized SARS-CoV-2 specific peptide as the substrate using the method of this invention.

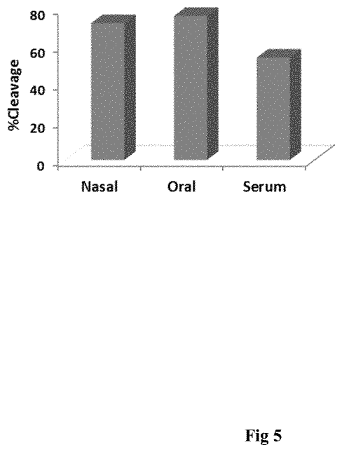

In this experiment, volunteer nasal and oral swab samples were collected according to the standard procedure of swab samples. The collected swab samples were released into 300 μl of PC Assay Buffer (Epigentek) by rotating the swab in the buffer for 30 seconds and then used for the assay with 30 μl of the sample solution. Human serum was obtained from Sigma and 5 ul of serum sample was directly used for assay. polystyrene 8-well strip microplate was coated with nickel (Ni-NTA). A SARS-CoV-2 specific substrate is tagged with polyhistidine at N-terminal and biotin at C-terminal and bound onto microplate wells through histidine/Ni-NTA at its N-terminal at the concentration of 10 ng/well. The strips were then incubated at 37° C. for 25 min. After wash with PBS-T for 3 times, the biological samples were added into the wells containing PC assay buffer consisted of sodium phosphate, sodium chloride and cesium chloride and incubated for 25 min. The wells were washed with PBS-T for 4 times. The cleavage of the substrate at S1/S2 site will remove the S2 part (C-terminal) of the substrate after washing, 100 ul of streptavidin-HRP at 1:5000 dilution was added into the wells for 15 min incubation. After washing 4 times, 100 μl of the color development solution containing TMB was added into the wells and wells were observed for 2-10 min for blue color appearance. The 1 M HCl or H₂504 solution was added to stop the color development and the optical density was measured with a microplate reader at a wavelength of 450 nm or measured with Spotxel® Reader installed on a smartphone. The results were shown in FIG. 5.

### Example 4

The experiment was carried out to examine the cleavage activity of purified PC or facilitated proteases and biological samples with complete trimeric form of full-length SARS-CoV-2 spike protein as the substrate using the method of this invention.

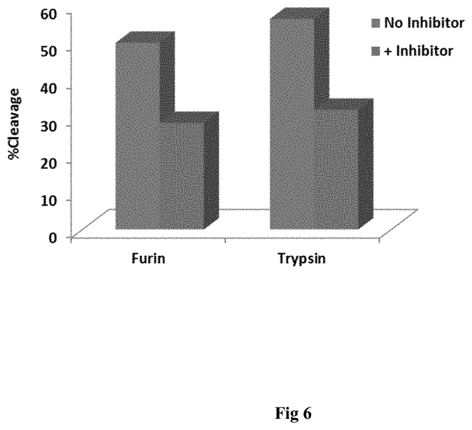

In this experiment, polystyrene 8-well strip microplate was coated with nickel (Ni-NTA). A full length of SARS-CoV-2 spike protein (S1+S2+EAC protein, from Seno Biological) is tagged with polyhistidine at C-terminal bound onto microplate wells through histidine/Ni-NTA at its N-terminal at the concentration of 50 ng/well. The strips were then incubated at 37° C. for 25 min. After wash with PBS-T for 3 times, the purified proprotein convertase furin, serine protease trypsin, plasmin and nasal swab sample were diluted to an indicated concentration with PC assay buffer consisted of sodium phosphate, sodium chloride and cesium chloride and were added into the wells for incubation for 25 min. The wells were washed with PBS-T for 3 times. The cleavage of the substrate at S1/S2 site will remove the S1 part (N-terminal) of the substrate after washing, 50 ul of HRP-conjugated anti-SARS-CoV-2 S1 RBD antibody solution diluted at 1:1000 was added into the wells and incubate for 1 h. After washing 4 times, 100 μl of the color development solution containing TMB was added into the wells and wells were observed for 2-10 min for blue color appearance. The 1 M HCl or H₂504 solution was added to stop the color development and the optical density was measured with a microplate reader at a wavelength of 450 nm or measured with Spotxel® Reader installed in a smartphone. The results were shown in the FIG. 6.

What is claimed is:

1. A method of detecting the presence of cleavage activity of an enzyme targeting to S1/S2 boundary cleavage region of SARS-CoV-2 spike protein in a sample comprising: (1) a substrate containing SARS-CoV-2 S1/S2 boundary cleavage region comprising: (a) said cleavage region consisting of at least one cleavage motif that can be cleaved by said enzyme; (b) labeling said substrate with at least an affinity moiety on at least one terminal region, which allows one terminal region of said substrate to be still attached to a solid-phase carrier containing an affinity partner after cleavage of said substrate; (2) at least an indicator molecule that is able to bind to at least an affinity moiety labeled on said substrate or one terminal region of the said substrate to indicate whether or not the substrate has been cleaved; and (3) fluorescent or color development of said indicator molecule and quantification of fluorescent or color intensity.

2. The method according to claim 1 wherein said substrate is a peptide containing SARS-CoV-2 S1/S2 boundary sequence with a length of 8-500 amino acids, or a recombinant full-length SARS-CoV-2 protein containing both receptor-binding subunit S1 and membrane fusion subunit S2.

3. The method according to claim 1 wherein said enzyme is a serine protease.

4. The method according to claim 1 wherein said sample is a serine protease-contained cell-free body fluids, or cells, or tissues.

5. The method according to claim 1 wherein said cleavage motif has an amino acid sequence Ala-Ser-Tyr-Gln-Thr-Gln-Thr-Asn-Ser-Pro-Arg-Arg-Ala-Arg-Ser-Val-Ala-Ser-Gln-Ser.

6. The method according to claim 1 wherein cleavage of the at least one cleavage motif generates two separate parts of the cleavage region, at least one part of which is removed from the solid-phase carrier after wash.

7. The method according to claim 1 wherein said affinity moiety is selected from an affinity substance group consisted of biotin, polyhistidine, antibody binding fragment, antibody binding fragment, IgG immunoglobulin domain, receptor molecule, GST, and nucleotide sequences.

8. The method according to claim 1 wherein said solid-phase carrier is polystyrene multi-well strip, or polystyrene slid, or magnetic beads.

9. The method according to claim 1 wherein said affinity partner is selected from an affinity molecule partner group consisted of streptavidin, neutravidin, captavdin, nickel, antigen binding fragment, protein A, protein G, receptor molecule ligand, GSH and complementary nucleotide sequences.

10. The method according to claim 1 wherein said indicator molecule is selected from a signal generation molecule group consisted of streptavidin-HRP, streptavidin-alkaline phosphatase (AP), streptavitin-fluorescent dyes, polyhistidine-HRP, polyhistidine-AP polyhistidine-fluorescent dyes.

11. A method for predicting the susceptibility to SARS-CoV-2 in a individual by detecting cleavage activity of an enzyme targeting to S1/S2 boundary cleavage region of SARS-CoV-2 spike protein in a sample comprising: (1) a substrate containing SARS-CoV-2 S1/S2 boundary cleavage region comprising: (a) said cleavage region consisting of at least one cleavage motif that can be cleaved by said enzyme; (b) labeling said substrate with at least an affinity moiety on at least one terminal region, which allows one terminal region of said substrate to still be attached to a solid-phase carrier after cleavage of said substrate; (2) bring said sample into contact with the said substrate for an appropriate time period; (3) bring an indicator molecule into contact with said substrate, which is able to bind to at least an affinity moiety labeled on said substrate or one terminal region of the said substrate to indicate whether or not the substrate has been cleaved; (4) fluorescent or color development of said indicator molecule and quantification of fluorescent or color intensity, wherein an increased cleavage activity in said sample indicate an increased susceptibility to SARS-CoV-2.

12. The method according to claim 1 wherein said substrate is a peptide containing SARS-CoV-2 S1/S2 boundary sequence with a length of 8-500 amino acids, or recombinant full length SARS-CoV-2 proteins containing both receptor-binding subunit S1 and membrane fusion subunit S2.

13. The method according to claim 11 wherein said sample is serine protease-contained cell-free body fluids, or cells, or tissues.

14. The method according to claim 11 wherein said cleavage motif has a sequence Ala-Ser-Tyr-Gln-Thr-Gln-Thr-Asn-Ser-Pro-Arg-Arg-Ala-Arg-Ser-Val-Ala-Ser-Gln-Ser.

15. The method according to claim 11 wherein cleavage of the at least one cleavage motif generates two separate parts of the cleavage region, at least one part of which is removed from the solid-phase carrier after wash.

16. A kit for detecting cleavage activity of an enzyme targeting to S1/S2 boundary cleavage region of SARS-CoV-2 spike protein in a sample comprising: (1) a solid-phase carrier pre-bound with a substrate containing SARS-CoV-2 S1/S2 boundary cleavage region and comprising: (a) said cleavage region consisting of at least one cleavage motif that can be cleaved by said enzyme; (b) said substrate that is labeled with at least an affinity moiety on at least one terminal region to allow one terminal region of said substrate to be still attached to a solid-phase carrier after cleavage of said substrate; (2) an indicator molecule solution into contact with said substrate by binding to an affinity moiety on one terminal region of said substrate to indicate whether or not the substrate has been cleaved; (3) a fluorescent or color development solution that can interact with said indicator molecule for quantification of fluorescent or color intensity.

17. The method according to claim 16 wherein said solid-phase carrier is a polystyrene multi-well strip.

18. The method according to claim 16 wherein said substrate is a peptide containing SARS-CoV-2 S1/S2 boundary sequence with a length of 8-500 amino acids, or recombinant full-length SARS-CoV-2 proteins containing both receptor-binding subunit S1 and membrane fusion subunit S2.

19. The method according to claim 16 wherein said affinity is selected from an affinity substance group consisted of biotin, polyhistidine, and antibody binding fragment.

20. The method according to claim 16 wherein said indicator molecule is selected from a signal generation molecule group consisted of streptavidin-HRP, streptavidin-AP, streptavitin-fluorescent dyes, polyhistidine-HRP, polyhistidine-AP, and polyhistidine-fluorescent dyes.


SEQUENCE LISTING (1 sequence)

SEQ ID NO: 1 - protein, 20 aa, Severe acute respiratory syndrome-related coronavirus
ASYQTQTNSPRRARSVASQS